(19) United States
(12) Patent Application Publication
(10) Pub. No.: US 2021/0205229 A1
(43) Pub. Date: Jul. 8, 2021

(54) MICROENCAPSULATED MODIFIED POLYNUCLEOTIDE COMPOSITIONS AND METHODS

(71) Applicant: MAYO FOUNDATION FOR MEDICAL EDUCATION AND RESEARCH, Rochester, MN (US)

(72) Inventors: Atta Behfar, Rochester, MN (US); Andre Terzic, Rochester, MN (US)

(21) Appl. No.: 17/056,648

(22) PCT Filed: May 23, 2019
(86) PCT No.: PCT/US2019/033705

§ 371 Date: Nov. 18, 2020

Related U.S. Application Data
(60) Provisional application No. 62/675,206, filed on May 23, 2018.

(51) Int. Cl. A61K 9/50; A61K 9/127; C12N 15/113

(52) U.S. Cl. CPC A61K 9/5036; C12N 15/113; A61K 9/501; A61K 9/1271

(57) ABSTRACT

A platform for introducing a heterologous polynucleotide into a cell so that the cell can express the transcription product of the heterologous polynucleotide includes compositions and methods. The compositions generally include an encapsulating agent and a polynucleotide encapsulated with the encapsulating agent. The encapsulating agent can include a metallic nanoparticle. The polynucleotide includes at least one modification to inhibit degradation of the polynucleotide in cytosol of a cell. In various embodiments, the polynucleotide encodes at least one therapeutic polypeptide or at least one therapeutic RNA. The method includes contacting a composition with a cell and allowing the cell to take up the composition.

### CROSS-REFERENCE TO RELATED APPLICATION

This application claims the benefit of U.S. Provisional Patent Application No. 62/675,206, filed May 23, 2018, which is incorporated herein by reference in its entirety.

### SUMMARY

This disclosure describes a platform and methods for introducing a heterologous polynucleotide into a cell so that the cell can express the transcription product of the heterologous polynucleotide.

Thus, in one aspect, this disclosure describes a composition that generally includes an encapsulating agent and a polynucleotide encapsulated with the encapsulating agent. The polynucleotide includes at least one modification to inhibit degradation of the polynucleotide in cytosol of a cell. In various embodiments, the polynucleotide encodes at least one therapeutic polypeptide or at least one therapeutic RNA.

In some embodiments, the encapsulating agent can include a metallic nanoparticle. In some of these embodiments, the metallic nanoparticle can include a plurality of metallic subunits. In some of these embodiments, the metallic subunits at least partially surround the polynucleotide; in other embodiments, the metallic subunits form a core structure.

In some embodiments, the polynucleotide can be an mRNA.

In another aspect, this disclosure describes a method of introducing a heterologous polynucleotide into a cell. Generally, the method includes contacting the cell with a pharmaceutical composition and allowing the cell to take up the pharmaceutical composition. The pharmaceutical composition generally includes an encapsulating agent and a heterologous polynucleotide encapsulated with the encapsulating agent. The heterologous polynucleotide includes at least one modification to inhibit degradation of the polynucleotide in cytosol of a cell.

In some embodiments, the encapsulating agent can include a metallic nanoparticle. In some of these embodiments, the metallic nanoparticle can include a plurality of metallic subunits. In some of these embodiments, the metallic subunits at least partially surround the polynucleotide; in other embodiments, the metallic subunits form a core structure.

In some embodiments, the heterologous polynucleotide encodes at least one therapeutic polypeptide or at least one therapeutic RNA.

In some embodiments, the heterologous polynucleotide can be an mRNA.

In some embodiments, the cell is in vivo.

In another aspect, this disclosure describes a method of introducing a therapeutic polypeptide or a therapeutic RNA into a cell. Generally, the method includes contacting the cell with a pharmaceutical composition, allowing the cell to take up the pharmaceutical composition, and allowing the cell to express the therapeutic polypeptide or therapeutic RNA. The therapeutic composition generally includes an encapsulating agent and a heterologous polynucleotide encapsulated with the encapsulating agent. The heterologous polynucleotide includes at least one modification to inhibit degradation of the heterologous polynucleotide when the heterologous polynucleotide is in cytosol of a cell. The heterologous polynucleotide encodes the therapeutic polypeptide or the therapeutic RNA.

In some embodiments, the encapsulating agent can include a metallic nanoparticle. In some of these embodiments, the metallic nanoparticle can include a plurality of metallic subunits. In some of these embodiments, the metallic subunits at least partially surround the polynucleotide; in other embodiments, the metallic subunits form a core structure.

In some embodiments, the cell is in vivo.

In some embodiments, the heterologous polynucleotide is an mRNA.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. mCherry protein expression in human dermal fibroblasts (HDFs), human cardiac fibroblasts (HCFs), and human embryonic kidney (HEK) cells. (A) Representative mCherry expression from HCFs and HEK cells. Scale bar=100 μm. (B) Quantitative changes in fluorescent intensity at the measurement time periods. Between 24 and 72 hours, intensity levels related to mCherry expression were more than two-fold over baseline. (C) Representative flow cytometry plot of HCFs and HEK cells after mCherry mRNA transfection. (D) Percent transfection efficiency of sorted HDFs, HCFs, and HEK cells at four hours and at 24 hours.

FIG. 2. mCherry protein expression in cardiomyocytes. (A) Rapid and sustained protein expression within primary cardiomyocytes. Scale bar=100 μm. (B) Quantification of the fluorescence intensity revealed maximum expression at 24 hours, declining in linear fashion for the subsequent six days. (C) Scatter plots of fluorescence intensity on the x-axis and sideward scattering signal on the y-axis revealed a consistent bimodal population following transfection with the transition revealing the number of transfected cells seen at four hours and 24 hours. Transfection efficiency was quantified and compared to mock transfected cells. The analysis of the four-hour and 24-hour transfection efficiency showed significant transfection efficiency at both the four-hour (˜20%) and 24-hour (43%) time points using flow cytometry. (D) GFP, mCherry, FLuc images show production of all three proteins in the same cell line. Scale bar=100 μm. (E) A comparison of EGFP and mCherry protein production in an identical set of cells; the merged image again demonstrates this synchronicity of expression.

FIG. 3. Fluorescent images of cardiomyocytes stained with anti-troponin antibody, anti-mCherry antibody, or subjected to SiR-Actin staining.

FIG. 4. Calcium imaging of transfected primary cardiomyocytes. (A) CAL-520 Am and mCherry staining of primary cardiomyocytes transfected with M³RNA. (B) Rhythmic and coordinated [Ca²⁺]ᵢ transients with synchronous rapid [Ca²⁺]ᵢ bursts during systole with its absence during diastole. (C) Plot of intracellular fluorescence intensity (Y-axis) versus duration of Ca²⁺ transients (X-axis).

FIG. 5. Electrical function of transfected primary cardiomyocytes. (A) mCherry-M³RNA transfected cells identified using fluorescence microscope. (B) A ramp pulse from −90 to +40 mV induced two typical inward current components that were different in voltage-gating properties. (C) The component with the peak value at ˜50 mV was typically sensitive to tetrodotoxin (TTX, 5 μM), a selective inhibitor of voltage-gated Na⁺ channels. The component at peak value ˜0 mV membrane potential was sensitive to nifedipine (20 μM), a voltage-dependent L-type Ca²⁺ channels inhibitor (I_{Ca}).

FIG. 6. Schematic diagrams of M³RNA structure and uptake by cells. (A) Exemplary embodiment using iron nanoparticles. Iron nanoparticles are coated with positively charged polymers. The positively charged nanoparticles encapsulate and interact with negatively charges mRNA to form M³RNA. (B) M³RNA enters the cell by endocytosis, the mRNA is released and translated.

FIG. 7. Bioluminescence and immunofluorescent study of M³RNA expression. (A) Bioluminescence imaging of cardiac-targeted expression of M³RNA within the heart. (B) Quantification of bioluminescence shown in (A). (C) mCherry protein expression in heart tissue injected with mCherry M³RNA compared to vehicle control (middle panels), with mCherry expression confirmed by anti-mCherry antibody in the green channel (left panels). Troponin antibody revealed mCherry expression in the cardiomyocytes. (D) Expression of GFP-M³RNA, mCherry-M³RNA, and FLuc-M³RNA in a single, multiple M³RNA species epicardial injection. GFP, mCherry and FLuc protein (using anti-FLuc antibody) expression overlapped in M³RNA injected rats (lower panels) versus no expression in sham (upper panels) (FIG. 7D).

FIG. 8. mCherry M³RNA encapsulated within a calcium-alginate solution provides targeted delivery of M³RNA to injured tissue in an acute porcine model of myocardial infarction. (A) An intracoronary bolus of ˜250 μg mCherry-M³RNA was infused into the left anterior descending coronary artery (LAD) using the distal opening of the infracting over-the-wire balloon. (B) Following intracoronary delivery, alginate was visualized to preferentially gel in the site of acute injury as monitored by intra-cardiac echocardiography (ICE). (C) The heart was harvested at 72 hours, flushed with chilled normal saline, sliced, and the sliced sections were imaged on Xenogen using mCherry filter, showing mCherry protein expression localized to the area of infarction. (D) Immunohistochemistry on 1-cm slices from areas of infarction versus non-infarcted regions featured higher mCherry staining.

FIG. 9. Exemplary modifications to mRNA in the M³RNA platform. (A) chemical structures of modified nucleotides pseudouridine and 5-methyl cytidine. (B) Schematic diagram of modifications to mRNA, showing incorporation of an anti-reverse cap analog (ARCA), modified nucleotides pseudouridine and 5-methyl cytidine, and polyadenylation (poly A) tail.

FIG. 10. In vivo FLuc mRNA expression. (A) No expression observed in control mouse receiving tail injection of only hydrodynamic solution. (B) FLuc expression was seen within two hours of tail vein injection of FLuc M²RNA. This expression was very transient, primarily in the liver and undetectable after 24 hours. (C) No expression observed in control mouse receiving null subcutaneous injection. (D) Subcutaneous delivery of the FLuc M³RNA resulted in 10-fold protein expression (vs. hydrodynamic) within two hours and was sustained for 72 hours. (E) Time course of FLuc M²RNA expression by tail vein injection. (F) Time course of FLuc M³RNA expression by subcutaneous injection.

FIG. 11. mCherry M³RNA expression 24 hours after subcutaneous injection.

FIG. 12. Expression of FLuc was seen in different tissues following administration. (A) Luciferase expression in the muscle at 24 hours. (B) Luciferase expression in the kidney at five hours. (C) Luciferase expression in the liver at four hours. (D) Luciferase expression in the eye at 24 hours. For intraocular injection, the left eye was used as a control.

FIG. 13. Quantitation of luciferase luminescence from IVIS images. (A) Image of intracardiac luciferase expression. (B) Expression of luciferase was sustained for a number of days after delivery with the expression levels peaking at 24 hours and declining afterwards.

FIG. 14. Quantitation of luciferase luminescence from IVIS images. Open chest image of intracardiac luciferase expression.

FIG. 15. Imaging of sliced heart sections on Xenogen using mCherry filter. (A) mCherry protein expression localized to the area of infarction when an alginate concentration of 1.5% was used. (B) Expression of mCherry was barely detectable in infarct tissue samples in the heart that received the same dose of mCherry M⁴RNA with an alginate concentration of 0.5%.

FIG. 16. Combination of M²RNA with microparticles coated with PEG and chitosan yields the putative M³RNA-Ig platform optimized for gene delivery in skeletal muscle.

FIG. 17. 3′ strategies to diminish the rate of mRNA degradation focuses on three putative platforms. The Pseudoknot mediates ribosomal read-through knocking off UPF1 molecules. The RNA stability element acts as a decoy to block UPF1 contact with the 3′UTR avoiding activation of the nonsense mediated decay (NMD). Poly(A) tail stem loop structures are used to diminish exosome-mediated mRNA degradation in constructs where a CAP/PABP independent IRES platform is used.

### DETAILED DESCRIPTION OF ILLUSTRATIVE EMBODIMENTS

This disclosure describes compositions and methods for achieving expression of heterologous proteins in vivo using a gene delivery system that involves microencapsulate modified polynucleotides referred to herein as M³RNA. The M³RNA platform can rapidly induce expression of a heterologous protein encoded by the M³RNA within a targeted tissue for a defined time horizon.

The M³RNA platform described herein overcomes challenges faced using viral-based or certain DNA-based therapeutics. RNA-based embodiments offer more rapid translation of the encoded protein than DNA-based therapeutics and does not require transfer into the target cell nucleus. The M³RNA platform overcomes challenges associated with conventional RNA-based therapeutics by providing delivery efficiency and functionality required to induce protein expression within targeted cell populations and/or tissues. The M³RNA platform overcomes challenges associated with virus-based therapeutics because it does not elicit an immune response against the M³RNA nanoparticles.

### Microencapsulated Modified RNA (M³RNA)

M³RNA is a unique platform by which to induce rapid expression of encoded genes into a broad array of tissues. In the context of the M³RNA platform, M³RNA refers to a modified microencapsulated polynucleotide; a naked modified polynucleotide (unencapsulated) is referred to as M²RNA; M⁴RNA refers to macroencapsulated polynucleotide (e.g., encapsulated with alginate), as described in more detail below. While the M³RNA nomenclature is derived from an exemplary embodiment described herein in which the polynucleotide is an mRNA (i.e., microencapsulated modified mRNA), the polynucleotide in an M³RNA can be any functional polynucleotide including, for example, mRNA, siRNA, miRNA, circularized RNA, or DNA. The M³RNA platform provides the ability to rapidly scale within a short timeframe and simultaneously deliver multiple gene constructs. Furthermore, unlike AAV and other viral gene-delivery technologies, the M³RNA platform avoids risk of immune reaction to the delivery system, allowing its repetitive use with different constructs. Embodiments in which the polynucleotide is an RNA limit risks associated with DNA-based therapeutics (e.g., integration or mutation).

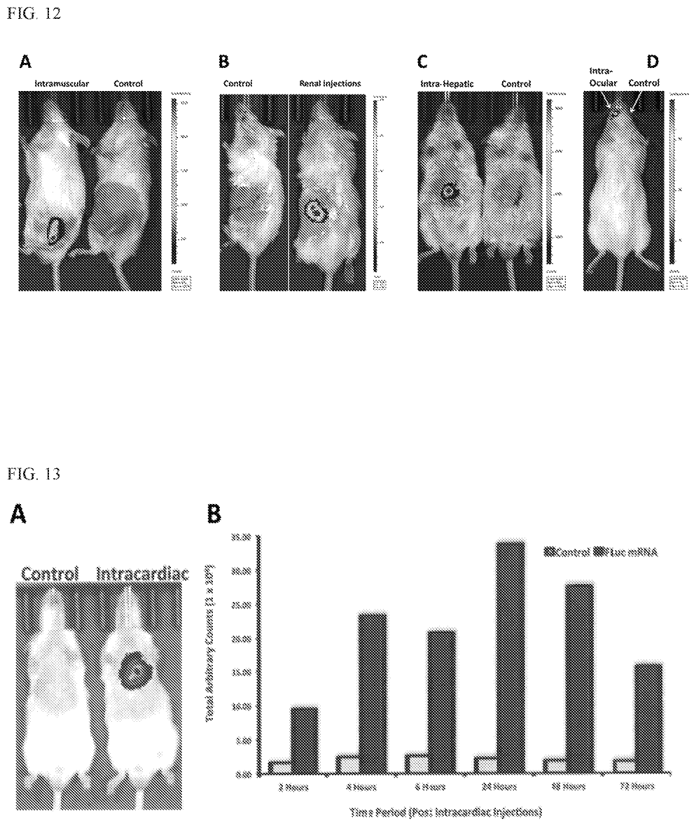

The M³RNA platform is compatible for use with any animal tissue, including human tissue. Moreover, the M³RNA platform is effective for transfecting “hard-to-transfect” primary cell phenotypes such as, for example, primary cardiomyocytes. As described in more detail below, transfection of primary cardiomyocytes using the M³RNA platform did not alter the structural or functional characteristics of cardiomyocytes. The M³RNA platform also is compatible with intramuscular delivery, providing high transfection efficiency comparable with results obtained with primary cardiomyocyte cultures. Generally, the M³RNA platform is compatible with tissue-specific delivery and expression of the protein encoded by the M³RNA (FIG. 12) and can be employed to transfect a broad range of cell types and/or tissues.

Generally, the M³RNA platform includes a polynucleotide (e.g., an mRNA), modified as described in more detail below, then encapsulated with or by an encapsulating agent (e.g., nanoparticle or lipid). As used herein, the polynucleotide is “encapsulated” with or by an encapsulating agent (e.g., a nanoparticle if it is in association with the encapsulating agent. Thus, it is not necessary for the mRNA to be enveloped, in whole or in part, in order to be “encapsulated” with or by the encapsulating agent. A polynucleotide may be in association with the encapsulating agent (e.g., a nanoparticle or a plurality of nanoparticles) by any suitable chemical or physical interaction including, but not limited to, a hydrogen bond, a disulfide bond, an ionic bond, or by being engulfed.

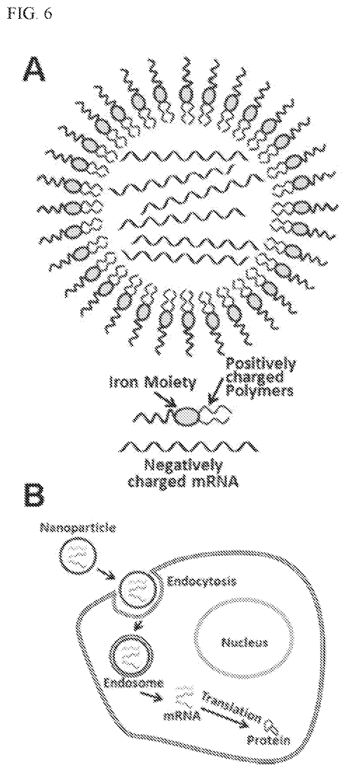

In some embodiments, schematically illustrated in FIG. 6A, the polynucleotide is at least partially enveloped by a nanoparticle that includes a plurality of metallic subunits, reflected in the exemplary embodiment illustrated in FIG. 6A as an “Iron Moiety.” The use of an iron-based metallic subunit is, however, merely exemplary. The metallic subunit may be formed from any suitable metal, as described in more detail below. In such an embodiment, at least some of the subunits may have a positively charged moiety (e.g., a positively charged polymer) attached to the metallic subunit. In some embodiments, the positively charged moiety can at least partially coat the subunit. Regardless of the identity of the positively charged moiety and the manner in which it attaches to the metallic subunit, the positively charged moiety can interact with the negatively charged polynucleotide. In the embodiment illustrated in FIG. 6A, a plurality of polymer-coated iron subunits forms a nanoparticle that surrounds a negatively-charged modified mRNA.

As illustrated in FIG. 6A, the metallic subunit can include a plurality of positively-charged moieties (e.g., illustrated as positively-charged polymers in FIG. 6A). When a plurality of positively-charged moieties is attached to a metallic subunit, each polymer attached to the metallic subunit may be the same or may be different than the other polymer or polymers attached to the metallic subunit. For example, in the embodiment illustrated in FIG. 6A, two positively-charged polymers are of one molecular species, which aligns to the inside of the nanoparticle formed by the plurality of metallic subunits. A different positively-charged polymer is also attached to the metallic subunit and aligns on the outside of the nanoparticle.

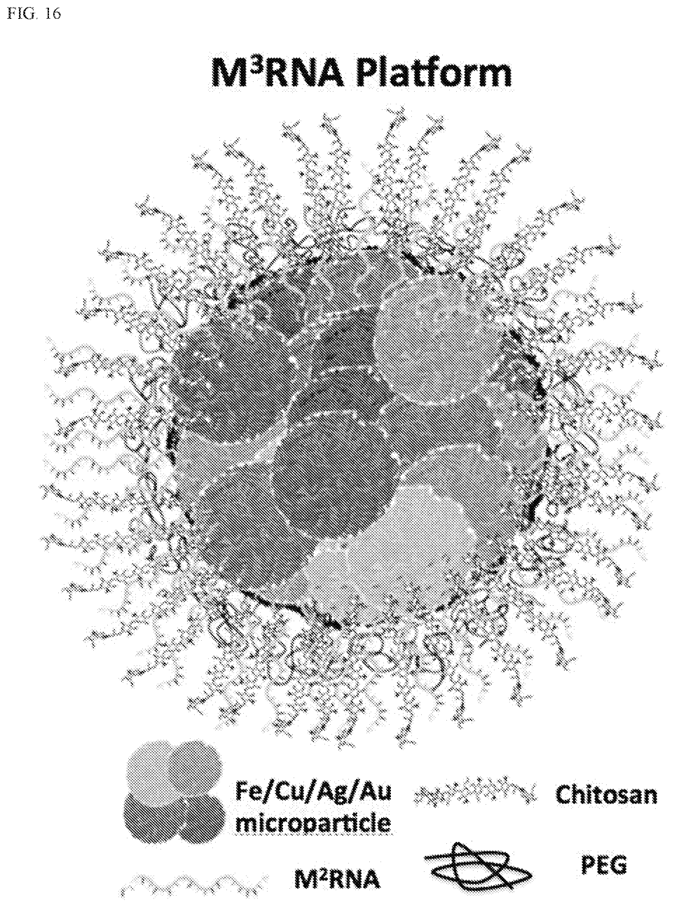

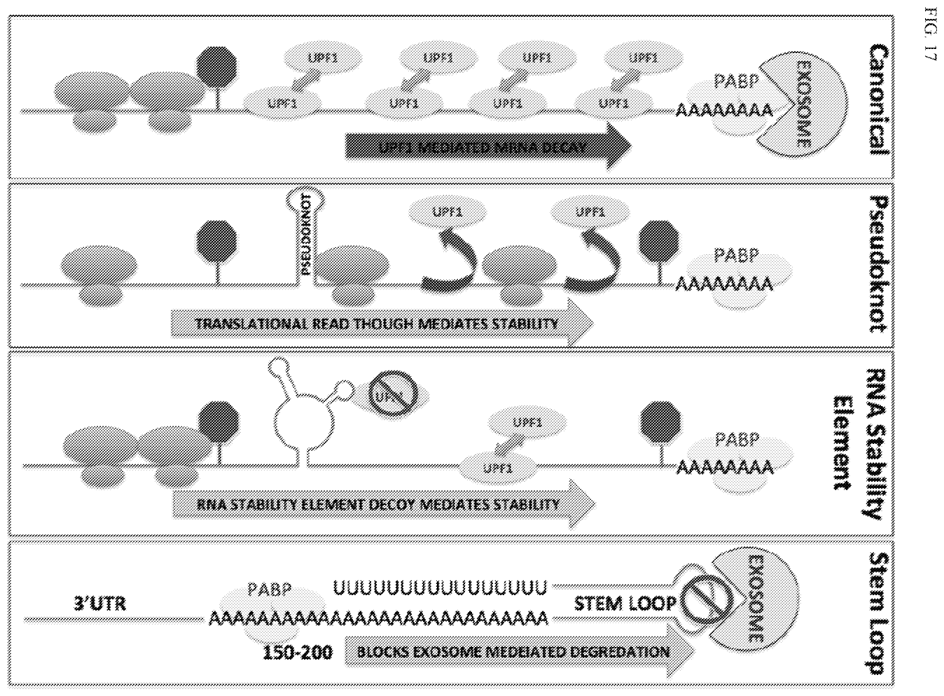

In another exemplary embodiment, schematically illustrated in FIG. 16, the polynucleotide interacts with a positively charged moiety—in the illustrated exemplary embodiment, chitosan—attached to the surface of a nanoparticle. The polynucleotide is considered to be encapsulated with or by the nanoparticle since the majority of the mass of the mRNA is within the outer diameter defined by the positively charged moiety attached to the surface of the nanoparticle core. Thus, the polynucleotide need not be enveloped, even in part, by the nanoparticle in order to be considered “encapsulated” with or by the nanoparticle.

The exemplary embodiment shown in FIG. 16 also illustrates that the nanoparticle can include a plurality of metallic subunits. As illustrated in FIG. 16, the metallic subunits can form a nanoparticle core rather than a shell, as is illustrated in FIG. 6A. FIG. 16 also illustrates that a metallic nanoparticle can include a heterogeneous mixture of metallic subunits. The nanoparticle can include a heterogeneous mixture of metallic subunits regardless of whether the subunits form a core, as shown in FIG. 16, or a shell, as shown in FIG. 6A.

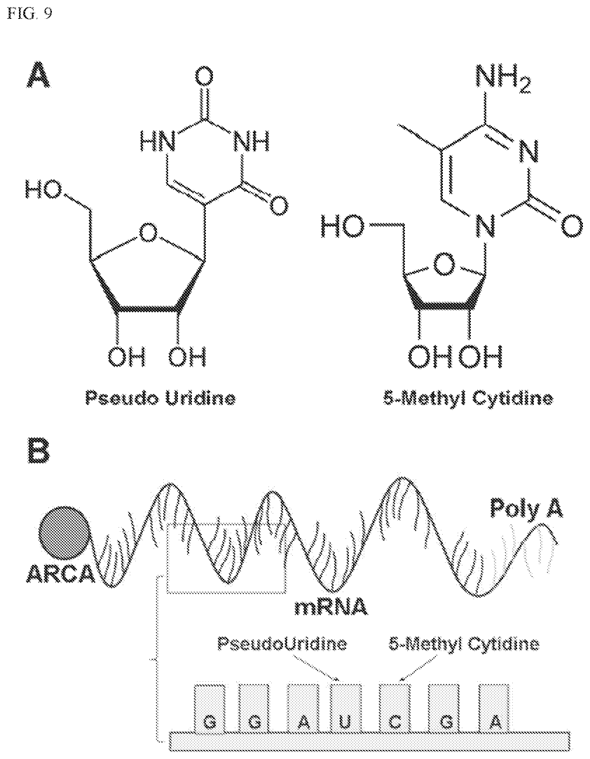

The M³RNA platform includes modifications to the encoding polynucleotide that can slow degradation of the M³RNA and/or can limit undesirable side effects of, for example, mRNA transfection. Such modifications include, for example, introducing one or more modified nucleotides such as, for example, 5′-methylcytidine in place of cytosine and/or pseudouridine (Ψ), dihydrouridine (D), or dideoxyuracil in place of uracil in an RNA. In some embodiments, at least one nucleotide is modified, e.g., at least 5, 10, 15, 20, 25, 50, 100 or more. In some embodiments, at least 1% of the cytosines and/or uracils are modified, e.g., at least 5%, 10%, 25%, 50% or more. Modified nucleotide triphosphates are readily abundant as GMP starting material and can be rapidly introduced using standard RNA synthesis techniques, providing significant molecular and translational advantage following delivery. Other strategies for extending the life of an mRNA in the cytosol involves interfering with the nonsense-mediated decay pathway. Exemplary suitable strategies include those illustrated in FIG. 17. Modifications to the mRNA also can include addition of an anti-reverse cap analog (ARCA cap) or a polyadenylated tail (FIG. 9B). In some embodiments, a modified mRNA can include one or more modified nucleotides, one or more pseudoknots, one or more RNA stability elements, one or more stem loops, an ARCA cap, and/or a polyadenylated tail in any combination.

The nanoparticle may be constructed of any suitable material including, but not limited to, metallic, organic (e.g., lipid-based), inorganic, or hybrid materials. Suitable metallic materials include, for example, iron, silver, gold, platinum, or copper. In some embodiments, cationic polymer nanoparticles are used to microencapsulate a modified polynucleotide. Cationic polymers have positively charged groups in their backbone to interact with negatively charged mRNA molecules to form neutralized, nanometer-sized complexes. Suitable cationic polymers include, for example, gelatin (Nitta Corp, JP). Suitable non-metallic materials include lipids. In some cases, a lipid-based nanoparticle may be complexed with other agents (e.g., polyethyleneimine (PEI)).

In certain embodiments, the nanoparticle can be an iron nanoparticle or include an iron subunit. In other embodiments, the nanoparticle can be comprised of a lipids or include a lipid component.

Also, modified nanoparticles can have controllable particle size and/or surface characteristics.

The nanoparticle that can be used in the M³RNA platform described herein can be any size suitable for the selected delivery method. A particle that can be used in the M³RNA platform can be from about 50 nm to about 12 μm in diameter, although, the compositions and methods described herein can include nanoparticles of a size outside of this range. Thus, the M³RNA platform can employ nanoparticles having a minimum diameter (or longest dimension) of at least 50 nm, at least 100 nm, at least 200 nm, at least 500 nm or at least 1 μm. The M³RNA platform can employ nanoparticles having a maximum diameter (or longest dimension) of no more than 12 μm, no more than 11.5 μm, no more than 11 μm, no more than 10.5 μm, no more than 10 μm, no more than 7.5 μm, no more than 5 μm, no more than 2 μm, no more than 1 μm, or no more than 500 nm. The M³RNA platform can employ nanoparticles having a diameter (or longest dimension) that falls within a range having endpoints defined by any minimum diameter listed above and any maximum diameter listed above that is greater than the minimum diameter. In certain exemplary embodiments, the nanoparticles may have a diameter of from about 50 nm to about 11.5 from about 100 nm to about 11 from about 200 nm to about 10.5 or from about 500 nm to about 10 μm) in diameter (or as measured across the longest dimension). For example, a particle that can be used in the M³RNA platform can be from about 50 nm to about 7.5 μm in diameter (or as measured across the longest dimension).

In some embodiments, the recited diameter range is an average diameter for a population of nanoparticles. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more of the particles in a population have the recited diameter.

In some cases, the size of the particle can be used to direct delivery of a particle to a target tissue (e.g., cardiac infarct bed). Human capillaries measure about 5 μm to 10 μm in diameter. Thus, a particle described herein having a diameter of from about 0.3 μm to about 12 μm can enter a capillary via the bloodstream, but be limited from exiting the capillary, where the biologics and/or an expressed polypeptide can diffuse into the capillary bed of a tissue (e.g., heart, dermal, lung, solid tumor, brain, bone, ligament, connective tissue structures, kidney, liver, subcutaneous, and vascular tissue).

The nanoparticle can be surface-modified for efficient interaction with the modified polynucleotide and/or to improve efficiency of delivery. Nanoparticles may be modified to introduce, for example, either a biopolymer or PEGylation that can, for example, increase blood circulation half-life. In particular embodiments, the surface of the nanoparticle may be modified with chitosan. Chitosan exhibits a cationic polyelectrolyte nature and therefore provides a strong electrostatic interaction with negatively charged DNA or RNA molecules. Moreover, chitosan carries primary amine groups that makes it a biodegradable, biocompatible, and non-toxic biopolymer that provides protection against DNase or RNase degradation. In some embodiments, the chitosan can have a viscosity average molecular weight of 5.3×10⁵ Daltons and/or an elemental composition of about 44% C, about 7% H, and about 8% N.

In alternative embodiments, the surface of the nanoparticles may be modified by PEGylation. The technique of covalently attaching the polyethylene glycol (PEG) to a given molecule, nanoparticle in this case, is a well-established method in targeted drug delivery systems. PEGylation involves the polymerization of multiple monomethoxy PEG (mPEG) that are represented as CH₃O—(CH₂—CH₂O)ₙ—CH₂—CH₂—OH, where n is from 100 to 5000. Introducing PEG molecules significantly increases the half-life of a nanoparticle due to its increased hydrophobicity, reduces glomerular filtration rate, and/or lowers immunogenicity due to masking of antigenic sites by forming protective hydrophilic shield. Suitable modifications include modifying the surface of the nanoparticles to possess 3000-4000 PEG molecules, which provides a suitable environment for the physical binding of DNA or RNA molecules.

The polynucleotide in the M³RNA can encode any suitable therapeutic polypeptide, any suitable inhibitory RNA, any suitable microRNA. In some cases, an M³RNA can include a plurality of polynucleotides, each of which can encode, independently of any other polynucleotide in the M³RNA, a therapeutic polypeptide or a therapeutic RNA (e.g., an inhibitory RNA or a microRNA).

The M³RNA platform can deliver a heterologous polynucleotide to any suitable cell type or cells of any suitable tissue. The delivery target (i.e., cell type or tissue) is not limiting. Thus, the M³RNA platform can be used to deliver a heterologous polynucleotide to, for example, a cardiac cell, a kidney cell, a liver cell, a skeletal muscle cell, an ocular cell, etc. to express a therapeutic polypeptide or a therapeutic RNA encoded by the heterologous polynucleotide in that target cell.

In some embodiments, the therapeutic polypeptide or therapeutic RNA encoded by the M³RNA polynucleotide can promote regenerating cardiac function and/or cardiac tissue.

Examples of polypeptides that can be useful for regenerating cardiac function and/or tissue include, without limitation, TNF-α, mitochondrial complex-1, resolvin-D1, NAP-2, TGF-α, ErBb3, VEGF, IGF-1, FGF-2, PDGF, IL-2, CD19, CD20, CD80/86, polypeptides described in WO 2015/034897, or an antibody directed against any of the foregoing polypeptides. For example, a human Nap-2 polypeptide can have the amino acid sequence set forth in, for example, National Center for Biotechnology Information (NCBI) Accession No. NP_002695.1 (GI No. 5473) and can be encoded by the nucleic acid sequence set forth in NCBI Accession No. NM_002704 (GI No. 5473). In some cases, a human TGF-α polypeptide can have the amino acid sequence set forth in NCBI Accession No. NP_003227.1 (GI No. 7039) and can be encoded by the nucleic acid sequence set forth in NCBI Accession No. NM_003236 (GI No. 7039). In some cases, a human ErBb3 polypeptide can have the amino acid sequence set forth in NCBI Accession No. NP_001005915.1 or NP_001973.2 (GI No. 2065) and can be encoded by the nucleic acid sequence set forth in NCBI Accession No. NM_001005915.1 or NM_001982.3 (GI No. 2065). For example, a human VEGF can have the amino acids set forth in NCBI Accession Nos. AAA35789.1 (GI: 181971), CAA44447.1 (GI: 37659), AAA36804.1 (GI: 340215), or AAK95847.1 (GI: 15422109), and can be encoded by the nucleic acid sequence set forth in NCBI Accession No. AH001553.1 (GI: 340214). For example, a human IGF-1 can have the amino acid sequence set forth in NCBI Accession No. CAA01954.1 (GI: 1247519) and can be encoded by the nucleic acid sequence set forth in NCBI Accession No. A29117.1 (GI: 1247518). For example, a human FGF-2 can have the amino acid sequence set forth in NCBI Accession No. NP_001997.5 (GI: 153285461) and can be encoded by the nucleic acid sequence set forth in NCBI Accession No. NM_002006.4 (GI: 153285460). For example, a human PDGF can have the amino acid sequence set forth in NCBI Accession No. AAA60552.1 (GI: 338209) and can be encoded by the nucleic acid sequence set forth in NCBI Accession No. AH002986.1 (GI: 338208). For example, a human IL-2 can have the amino acid sequence set forth in NCBI Accession No. AAB46883.1 (GI: 1836111) and can be encoded by the nucleic acid sequence set forth in NCBI Accession No. 577834.1 (GI: 999000). For example, a human CD19 can have the amino acid sequence set forth in NCBI Accession No. AAA69966.1 (GI: 901823) and can be encoded by the nucleic acid sequence set forth in NCBI Accession No. M84371.1 (GI: 901822). For example, a human CD20 can have the amino acid sequence set forth in NCBI Accession No. CBG76695.1 (GI: 285310157) and can be encoded by the nucleic acid sequence set forth in NCBI Accession No. AH003353.1 (GI: 1199857). For example, a human CD80 can have the amino acid sequence set forth in NCBI Accession No. NP_005182.1 (GI: 4885123) and can be encoded by the nucleic acid sequence set forth in NCBI Accession No. NM_005191.3 (GI: 113722122), and a human CD86 can have the amino acid sequence set forth in NCBI Accession No. AAB03814.1 (GI: 439839) and can be encoded by the nucleic acid sequence set forth in NCBI Accession No. CR541844.1 (GI: 49456642). For example, a polypeptide that can be useful for regenerating cardiac function and/or tissue can be an antibody directed against TNF-α, mitochondrial complex-1, or resolvin-D1. In some cases, an M³RNA can encode NAP-2 and/or TGF-α.

In some cases, an M³RNA can encode one or more inhibitory RNAs useful, for example, to treat a mammal experiencing a major adverse cardiac event (e.g., acute myocardial infarction) and/or a mammal at risk of experiencing a major adverse cardiac event (e.g., patients who underwent PCI for STEMI). For example, an M³RNA can encode an inhibitory RNA inhibiting and/or reducing expression of one or more of the following polypeptides: eotaxin-3, cathepsin-S, DK-1, follistatin, ST-2, GRO-α, IL-21, NOV, transferrin, TNFαRI, TNFαRII, angiostatin, CCL25, ANGPTL4, MMP-3, and polypeptides described in WO 2015/034897. For example, a human eotaxin-3 polypeptide can have an amino acid sequence set forth in, for example, NCBI Accession No: No. NP_006063.1 (GI No. 10344) and can be encoded by the nucleic acid sequence set forth in NCBI Accession No. NM_006072 (GI No. 10344). In some cases, a human cathepsin-S can have the amino acid sequence set forth in NCBI Accession No. NP_004070.3 (GI No. 1520) and can be encoded by the nucleic acid sequence set forth in NCBI Accession No. NM_004079.4 (GI No. 1520). In some cases, a human DK-1 can have the amino acid sequence set forth in NCBI Accession No. NP_036374.1 (GI No. 22943) and can be encoded by the nucleic acid sequence set forth in NCBI Accession No. NM_012242 (GI No. 22943). In some cases, a human follistatin can have then amino acid sequence set forth in NCBI Accession No. NP_037541.1 (GI No. 10468) and can be encoded by the nucleic acid sequence set forth in NCBI Accession No. NM_013409.2 (GI No. 10468). In some cases, a human ST-2 can have the amino acid sequence set forth in NCBI Accession No. BAA02233 (GI No. 6761) and can be encoded by the nucleic acid sequence set forth in NCBI Accession No D12763.1 (GI No 6761). In some cases, a human GRO-α polypeptide can have the amino acid sequence set forth in NCBI Accession No. NP_001502.1 (GI No. 2919) and can be encoded by the nucleic acid sequence set forth in NCBI Accession No. NM_001511 (GI No. 2919). In some cases, a human IL-21 can have the amino acid sequence set forth in NCBI Accession No. NP_068575.1 (GI No. 59067) and can be encoded by the nucleic acid sequence set forth in NCBI Accession No. NM_021803 (GI No. 59067). In some cases, a human NOV polypeptide can have the amino acid sequence set forth in NCBI Accession No. NP_002505.1 (GI No. 4856) and can be encoded by the nucleic acid sequence set forth in NCBI Accession No. NM_002514 (GI No. 4856). In some cases, a human transferrin polypeptide can have the amino acid sequence set forth in NCBI Accession No. NP_001054.1 (GI No. 7018) and can be encoded by the nucleic acid sequence set forth in NCBI Accession No. NM_001063.3 (GI No. 7018). In some cases, a human TIMP-2 polypeptide can have the amino acid sequence set forth in NCBI Accession No. NP_003246.1 (GI No. 7077) and can be encoded by the nucleic acid sequence set forth in NCBI Accession No. NM_003255.4 (GI No. 7077). In some cases, a human TNFαRI polypeptide can have the amino acid sequence set forth in NCBI Accession No. NP_001056.1 (GI No. 7132) and can be encoded by the nucleic acid sequence set forth in NCBI Accession No. NM_001065 (GI No. 7132). In some cases, a human TNFαRII polypeptide can have the amino acid sequence set forth in NCBI Accession No. NP_001057.1 (GI No. 7133) and can be encoded by the nucleic acid sequence set forth in NCBI Accession No. NM_001066 (GI No. 7133). In some cases, a human angiostatin polypeptide can have the amino acid sequence set forth in NCBI Accession No. NP_000292 (GI No. 5340) and can be encoded by the nucleic acid sequence set forth in NCBI Accession No. NM_000301 (GI No. 5340). In some cases, a human CCL25 polypeptide can have the amino acid sequence set forth in NCBI Accession No. NP_005615.2 (GI No. 6370) and can be encoded by the nucleic acid sequence set forth in NCBI Accession No. NM_005624 (GI No. 6370). In some cases, a human ANGPTL4 polypeptide can have the amino acid sequence set forth in NCBI Accession No. NP_001034756.1 or NP_647475.1 (GI No. 51129) and can be encoded by the nucleic acid sequence set forth in NCBI Accession No. NM_001039667.1 or NM_139314.1 (GI No. 51129). In some cases, a human MMP-3 polypeptide can have the amino acid sequence set forth in NCBI Accession No. NP_002413.1 (GI No. 4314) and can be encoded by the nucleic acid sequence set forth in NCBI Accession No. NM_002422 (GI No. 4314).

In some cases, an M³RNA can encode one or more nucleotides that modulate (e.g., mimics or inhibits) a microRNA involved in cardiac regenerative potency. For example, an M³RNA can encode an agomiR that mimics a miRNA to augment cardiac regenerative potency. For example, an M³RNA can encode an antagomiRs that inhibits a miRNA to augment cardiac regenerative potency. Examples of miRNAs involved in cardiac regenerative potency include, without limitation, miR-127, miR-708, miR-22-3p, miR-411, miR-27a, miR-29a, miR-148a, miR-199a, miR-143, miR-21, miR-23a-5p, miR-23a, miR-146b-5p, miR-146b, miR-146b-3p, miR-2682-3p, miR-2682, miR-4443, miR-4443, miR-4521, miR-4521, miR-2682-5p, miR-2682, miR-137.miR-137, miR-549.miR-549, miR-335-3p, miR-335, miR-181c-5p, miR-181c, miR-224-5p, miR-224, miR-3928, miR-3928, miR-324-5p, miR-324, miR-548h-5p, miR-548h-1, miR-548h-5p, miR-548h-2, miR-548h-5p, miR-548h-3, miR-548h-5p, miR-548h-4, miR-548h-5p, miR-548h-5, miR-4725-3p, miR-4725, miR-92a-3p, miR-92a-1, miR-92a-3p, miR-92a-2, miR-134, miR-134, miR-432-5p, miR-432, miR-651, miR-651, miR-181a-5p, miR-181a-1, miR-181a-5p, miR-181a-2, miR-27a-5p, miR-27a, miR-3940-3p, miR-3940, miR-3129-3p, miR-3129, miR-146b-3p, miR-146b, miR-940, miR-940, miR-484, miR-484, miR-193b-3p, miR-193b, miR-651, miR-651, miR-15b-3p, miR-15b, miR-576-5p, miR-576, miR-377-5p, miR-377, miR-1306-5p, miR-1306, miR-138-5p, miR-138-1, miR-337-5p, miR-337, miR-135b-5p, miR-135b, miR-16-2-3p, miR-16-2, miR-376c.miR-376c, miR-136-5p, miR-136, let-7b-5p, let-7b, miR-377-3p, miR-377, miR-1273g-3p, miR-1273g, miR-34c-3p, miR-34c, miR-485-5p, miR-485, miR-370.miR-370, let-7f-1-3p, let-7f-1, miR-3679-5p, miR-3679, miR-20a-5p, miR-20a, miR-585.miR-585, miR-3934, miR-3934, miR-127-3p, miR-127, miR-424-3p, miR-424, miR-24-2-5p, miR-24-2, miR-130b-5p, miR-130b, miR-138-5p, miR-138-2, miR-769-3p, miR-769, miR-1306-3p, miR-1306, miR-625-3p, miR-625, miR-193a-3p, miR-193a, miR-664-5p, miR-664, miR-5096.miR-5096, let-7a-3p, let-7a-1, let-7a-3p, let-7a-3, miR-15b-5p, miR-15b, miR-18a-5p, miR-18a, let-7e-3p, let-7e, miR-1287.miR-1287, miR-181c-3p, miR-181c, miR-3653, miR-3653, miR-15b-5p, miR-15b, miR-1, miR-1-1, miR-106a-5p, miR-106a, miR-3909.miR-3909, miR-1294.miR-1294, miR-1278, miR-1278, miR-629-3p, miR-629, miR-340-3p, miR-340, miR-200c-3p, miR-200c, miR-22-3p, miR-22, miR-128, miR-128-2, miR-382-5p, miR-382, miR-671-5p, miR-671, miR-27b-5p, miR-27b, miR-335-5p, miR-335, miR-26a-2-3p, miR-26a-2, miR-376b. miR-376b, miR-378a-5p, miR-378a, miR-1255a, miR-1255a, miR-491-5p, miR-491, miR-590-3p, miR-590, miR-32-3p, miR-32, miR-766-3p, miR-766, miR-30c-2-3p, miR-30c-2, miR-128.miR-128-1, miR-365b-5p, miR-365b, miR-132-5p, miR-132, miR-151b. miR-151b, miR-654-5p, miR-654, miR-374b-5p, miR-374b, miR-376a-3p, miR-376a-1, miR-376a-3p, miR-376a-2, miR-149-5p, miR-149, miR-4792.miR-4792, miR-1.miR-1-2, miR-195-3p, miR-195, miR-23b-3p, miR-23b, miR-127-5p, miR-127, miR-574-5p, miR-574, miR-454-3p, miR-454, miR-146a-5p, miR-146a, miR-7-1-3p, miR-7-1, miR-326.miR-326, miR-301a-5p, miR-301a, miR-3173-5p, miR-3173, miR-450a-5p, miR-450a-1, miR-7-5p, miR-7-1, miR-7-5p, miR-7-3, miR-450a-5p, miR-450a-2, miR-1291, miR-1291, miR-7-5p, miR-7-2, and miR-17-5p, miR-17.

The M³RNA may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutically active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with M³RNA without causing any undesirable biological effects or interacting in a deleterious manner with any of the other components of the pharmaceutical composition in which it is contained.

The M³RNA may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release. In some embodiments, the M³RNA may be administered directly to cardiac tissue such as, for example, intracardiac injection, intracoronary delivery, delivery to the coronary sinus, or delivery to the Thebesian vein circulation.

Thus, the M³RNA may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the M³RNA into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of M³RNA administered can vary depending on various factors including, but not limited to, the weight, physical condition, and/or age of the subject, the target cell or tissue to which the M³RNA is being delivered and/or the route of administration. Thus, the absolute amount of M³RNA included in a given unit dosage form can vary widely, and depends upon factors such as the species, age, weight and physical condition of the subject, and/or the method of administration. Accordingly, it is not practical to set forth generally the amount that constitutes an amount of M³RNA effective for all possible applications. Those of ordinary skill in the art, however, can readily determine the appropriate amount with due consideration of such factors.

In some embodiments, the method can include administering sufficient M³RNA to provide a dose of, for example, from about 100 ng/kg to about 50 mg/kg to the subject, although in some embodiments the methods may be performed by administering M³RNA in a dose outside this range. In some of these embodiments, the method includes administering sufficient M³RNA to provide a dose of from about 10 μg/kg to about 5 mg/kg to the subject, for example, a dose of from about 100 μg/kg to about 1 mg/kg.

In some embodiments, M³RNA may be administered, for example, from a single dose to multiple doses per day or per week, although in some embodiments the method can be performed by administering M³RNA at a frequency outside this range. When multiple doses are used within a certain period, the amount of each dose may be the same or different. For example, a dose of 1 mg per day may be administered as a single dose of 1 mg, two 0.5 mg doses, or as a first dose of 0.75 mg followed by a second dose of 0.25 mg. Also, when multiple doses are used within a certain period, the interval between doses may be the same or be different.

In certain embodiments, M³RNA may be administered from about once per month to about five times per week.

### M³RNA Transfection in Multiple Cell Lines

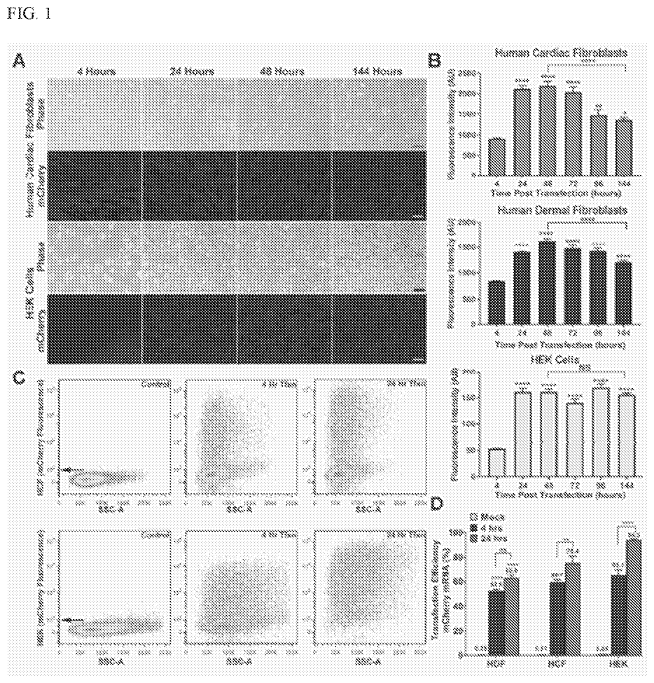

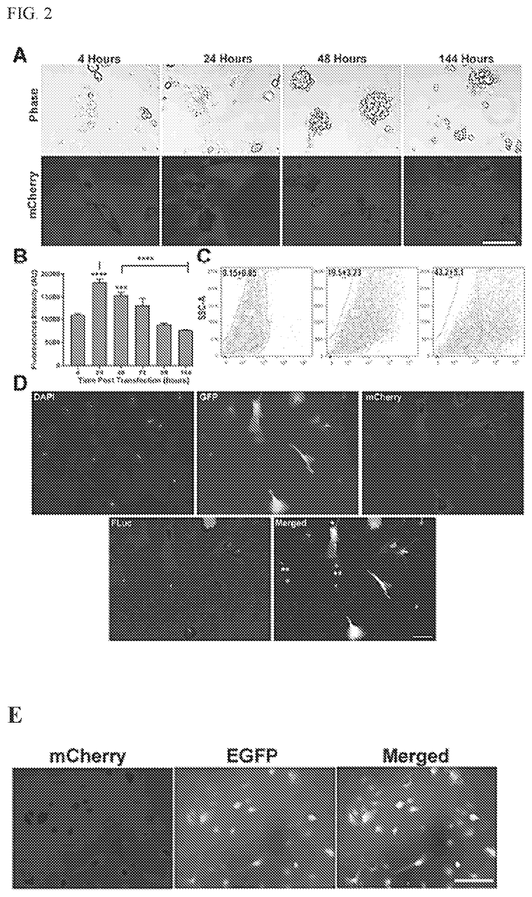

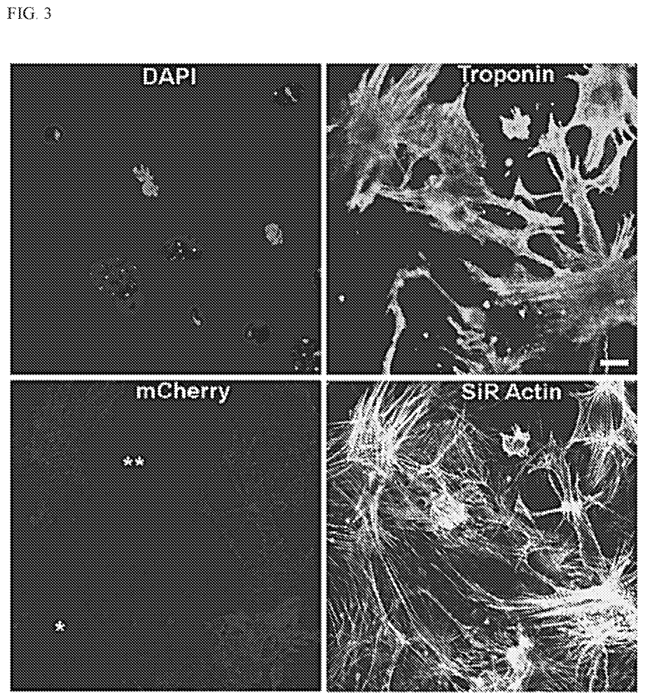

An exemplary model M³RNA including an mRNA that encodes a fluorescent protein (mCherry) was transfected into human dermal fibroblasts (HDF), human cardiac fibroblasts (HCF), and human embryonic kidney cells (HEK293) cells. Fluorescent protein expression was imaged live in 37° C. humidified chamber with 5% CO₂. mCherry protein expression was detected in as little as two hours and was reproducibly quantifiable at four hours. Fluorescent images of HCF and HEK293 cells (FIG. 1A) show rapid mCherry protein expression that was sustained for six days. Simultaneous delivery of M³RNAs encoding mCherry and GFP resulted in co-expression (FIG. 2E). Quantification of fluorescence intensity within three different cell lines (>10 fields of cells/time period/cell line) noted an increase in intensity over the initial 24-48 hours, which remained steady in at least the HEK cells (FIG. 1B). Since protein expression peaked at 24 hours, transfection efficiency was measured at four hours and 24 hours using flow cytometry. Scatter plots of fluorescence intensity on the x-axis and sideward scattering signal on the y-axis revealed a consistent bimodal population following transfection (FIG. 1C) with the transition revealing the number of transfected cells seen at four hours and at 24 hours. Transfection efficiency was quantified and compared to mock transfected cells (FIG. 1D). Note the high transfection efficiency at 24-hour time point, especially in the HEK population.

### M³RNA Transfection in Rat Neonatal Primary Cardiomyocytes

The M³RNA platform was then used to transfect hard-to-transfect primary cardiomyocytes. Cardiomyocyte-enriched cultures, following documentation of a synchronous beating pattern, were transfected with mCherry M³RNA. Fluorescence images at four hours up to six days were acquired. Representative images showed rapid and sustained protein expression within primary cardiomyocytes (FIG. 2A). Quantification of the fluorescence intensity revealed maximum expression at 24 hours and fluorescence remained detectable for six days (FIG. 2B). Significant transfection efficiency was seen at four hours (˜20%) and 24 hours (43%) using flow cytometry from two independent experiments (FIG. 2C). Multi-gene transfection showed simultaneous expression of three proteins (EGFP, mCherry, and Firefly Luciferase) within the same cardiomyocytes (FIG. 2D).

Transfection does not Alter Cardiomyocyte Structure and Function

To test that transfection of primary cardiomyocytes does not alter their structural integrity, cardiomyocytes were transfected with mCherry M³RNA and stained with cardiac-specific troponin antibody and SiR-Actin staining. Actin staining was used to differentiate cardiomyocytes from fibroblasts. No significant differences between the cardiomyocytes specific troponin staining were identified in the transfected versus non-transfected cells, indicating intact structural integrity of cardiomyocytes.

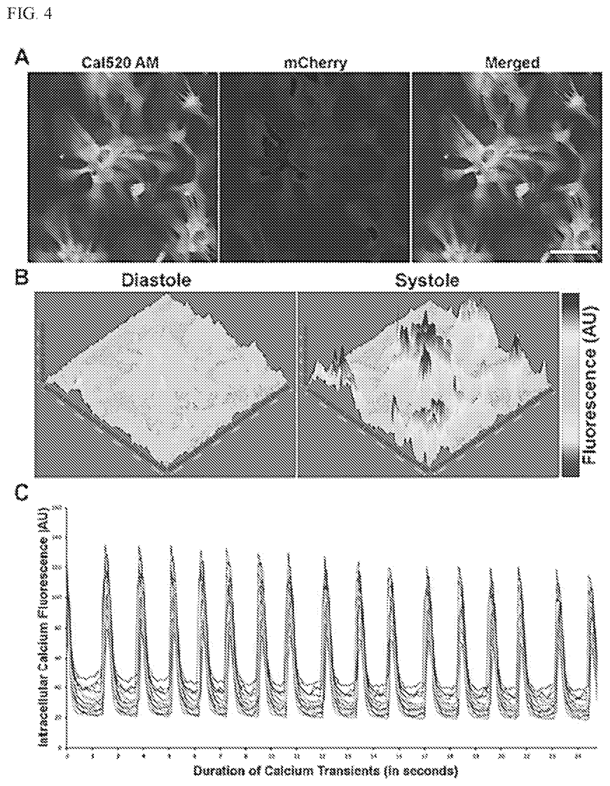

To determine, if the transfection alters the central electrical properties of transfected cells, two intrinsic functional parameters of primary cardiomyocytes were compared: 1) calcium channel transients and 2) voltage-current relationships. [Ca²⁺]ᵢ (Intracellular calcium) transients from primary cardiomyocytes were recorded using the free intracellular Ca²⁺ binding dye CAL-520 AM (AAT Bioquest, Inc., Sunnyvale, Calif.). Primary cardiomyocytes meeting the beating pattern criterion were transfected with mCherry M³RNA. To image Ca²⁺ transients using CAL-520 AM, fields featuring both transfected and non-transfected cells were selected using the mCherry filter (FIG. 4A). Robust [Ca²⁺]ᵢ transients were observed in the primary cardiomyocytes cultures having mCherry expression. Representative annotation of fluorescence intensity created at systole and diastole revealed the rhythmic and coordinated (in both transfected and non-transfected cells) [Ca²⁺]ᵢ transients with synchronous rapid [Ca²⁺]ᵢ bursts during systole with its absence during diastole (FIG. 4B). Regions of interest were created from transfected and non-transfected cells (FIG. 4A) and intracellular fluorescence intensity (Y-axis) versus duration of Ca²⁺ transients (X-axis) were plotted (FIG. 4C). Note the similar [Ca²⁺]ᵢ transients in transfected and non-transfected cells.

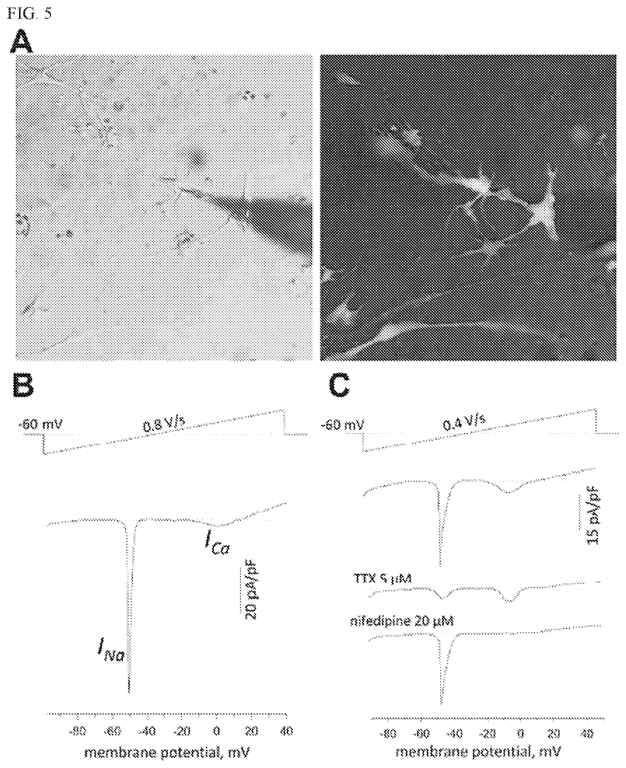

To test the electrical function of transfected primary cardiomyocytes, cardiomyocyte excitability and contraction were tested. Beating cells were transfected overnight using mCherry M³RNA and transfected cells were identified using the fluorescence microscope (FIG. 5A). To discriminate inward currents components responsible for the cell excitation the transfected neonatal cardiomyocytes were exposed to the ramp stimulation protocol in the whole-cell patch-clamp mode of recordings. Under such conditions, a ramp pulse from −90 to +40 mV induced two typical inward current components that were different in voltage-gating properties (FIG. 5B). The first component with the peak value at ˜50 mV was typically sensitive to tetrodotoxin (TTX, 5 μM), a selective inhibitor of voltage-gated Na⁺ channels (FIG. 5C). The second component at peak value ˜0 mV membrane potential, sensitive to nifedipine, a voltage-dependent L-type Ca²⁺ channels inhibitor (I_{Ca}). Thus, the obtained voltage-current relationships revealed intact profile of I_{Na} and I_{Ca} current components under mCherry transfection.

### M³RNA-FLuc Myocardial Injection Induces Prompt Protein Expression

Rapid expression in primary cardiomyocytes under in vivo conditions was confirmed using direct myocardial injections of nanoparticle-based FLuc M³RNA into the left ventricle of FVB mice. For in vivo studies, nanoparticles (˜100 nm) coated with positively charged biological polymers were used as carriers of mRNA. The positively charged nanoparticles enveloped negatively-charged mRNA molecules (FIG. 6A). Upon in vivo administration of the M³RNA, nanoparticles enter the cells by endocytosis and release mRNA molecules for translation. Nanoparticles composed of iron subunits get degraded and released iron enters normal iron metabolic pathway. FLuc is used to determine protein expression kinetics in live animals.

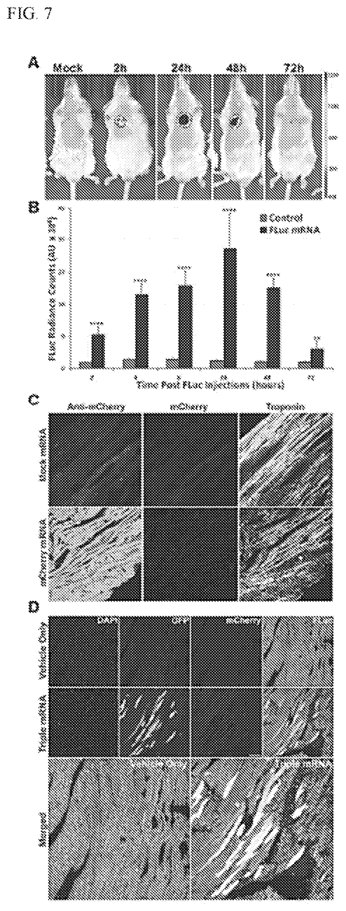

Bioluminescence imaging documented cardiac targeted expression within the heart in as early as two hours post injection, increasing nearly 3.5 times in 24 hours and fading to nearly background levels by 72 hours (FIGS. 7A and 7B). No off-target transfection was observed as signal was detected only in the heart area (FIG. 7A). Further, serial sections 24 hours after mCherry-M³RNA intracardiac injection revealed significant mCherry protein expression in heart tissue injected with mCherry mRNA compared to vehicle control (FIG. 7C, middle bottom panel), with mCherry expression confirmed by anti-mCherry antibody in the green channel (FIG. 7C, left bottom panel). Troponin antibody reveled mCherry expression in the cardiomyocytes and note (*) expression of mCherry in non-cardiomyocytes areas as well. Finally, multiple gene expression with a single epicardial injection was performed using GFP-M³RNA, mCherry-M³RNA, and FLuc-M³RNA versus vehicle only in rat hearts. FLuc imaging can be performed on live animals; therefore, FLuc expression was confirmed within mouse heart at 24 hours using Xenogen and the animal was then sacrificed, and heart tissues were processed for immunofluorescence (IF) analysis. IF revealed GFP, mCherry and FLuc protein (using anti-FLuc antibody) expression overlapped in M³RNA injected rats (lower panels) versus no expression in sham (upper panels) (FIG. 7D).

Targeted Expression of mCherry M³RNA in a Porcine Model of Acute MI

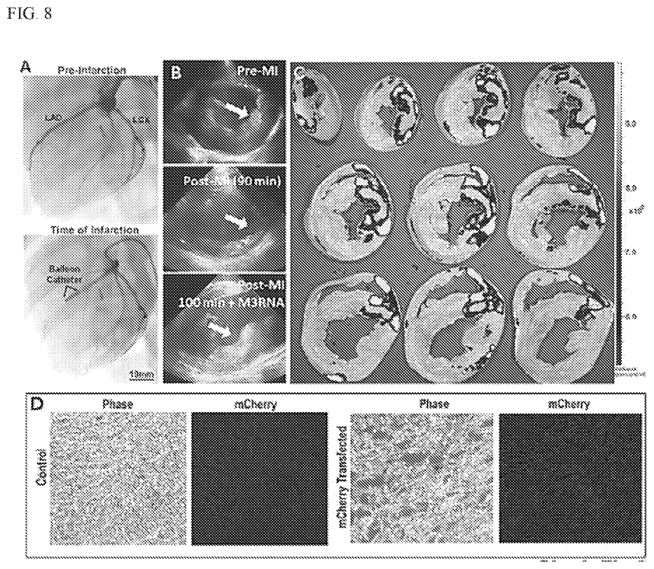

mCherry M³RNA was encapsulated within a calcium-alginate solution. Using an acute porcine model of myocardial infarction (FIG. 8A), an intracoronary bolus of ˜250 μg mCherry-M³RNA was infused into the left anterior descending coronary artery (LAD) using the distal opening of the infracting over-the-wire balloon. Following intracoronary delivery, alginate was visualized to preferentially gel in the site of acute injury as monitored by intra-cardiac echocardiography (ICE; FIG. 8B). The heart was harvested at 72 hours, flushed with chilled normal saline and sliced using a ProCUT sampling tool. Imaging of sliced heart sections on Xenogen using mCherry filter showed significant mCherry protein expression localized to the area of infarction (FIG. 8C). Immunohistochemistry on 1-cm slices from areas of infarction versus non-infarcted regions featured significantly higher mCherry staining (FIG. 8D), confirming targeted induction of protein expression within the injured portion of the heart.

Gene therapy is a promising strategy for treatment and regeneration in, for example, cardiovascular diseases. Some clinical scenarios require gene expression or gene editing to reverse the course of disease. Such clinical scenarios include, for example, clotting disorders, enzymatic deficiency, or gene mutation. However, within the healthy population, an adverse inflammatory response to acute events may result in tissue non-healing or chronic injury. DNA and viral vectors are great tools for treating diseases where long term expression of an encoded protein is required. An mRNA vector may be more appropriate where transient expression may be preferable, such as in attenuating acute inflammation and/or CRISPR-mediated genome editing, where off-target events are undesirable. RNA vectors provide certain advantages over DNA-based and viral-based therapeutics. For example, RNA vectors present almost no risk of genome integration compared to DNA vectors, invoke no immune response compared to viral vectors, and can initiate rapid and transient protein expression compared to both DNA-based and viral-based therapeutics.

This disclosure describes a novel M³RNA-based approach to induce rapid expression that is compatible across multiple cell lines, including primary cardiomyocytes, heart, and acutely injured myocardium. This platform showed controlled expression kinetics in multiple cell lines and primary cells, with transfection having little or no impact on the structural and functional properties of primary cardiomyocytes. Myocardial injection of M³RNA encoding model reporter proteins FLuc, mCherry, and GFP reproducibly induced rapid and consistent protein expression within heart tissue. Furthermore, this approach was found flexible enough for simultaneous delivery of multiple genes into heart tissue and could be targeted into acutely injured tissue in a porcine model of myocardial infarction. While illustrated above in the context of an exemplary embodiment in which the M³RNA platform is used to transfect cardiac tissue, the M³RNA platform may be used to transfect cells of other tissues such as, for example, fibroblasts, skeletal muscle, kidney, liver, and/or ocular tissues.

The M³RNA-based platform described herein can improve patient outcomes. For example, during acute myocardial infarction, a rapid sequence of molecular events occurs during injury and following reperfusion that ultimately culminate in damage to tissue. Injury can be fully aborted with rapid percutaneous coronary intervention (PCI) if the patient presents within a very short period of time (<90 minutes). However, in those that present >90 min to <12 hours, PCI is still indicated but the scope of damage to myocardium becomes increasingly worse due to ischemia and hypoxia. Indeed, in most individuals, restoration of blood flow even after the initial 90 minutes results in recovery of myocardial function and restoration or organ performance to near normal. However, in about 30% of the population, severe loss of myocardium occurs despite reperfusion. Efforts to mitigate this phenomenon have been focused on anti-platelet agents and neurohormonal antagonism. However, a compendium of recent evidence suggests that a deregulation of the inflammatory response to injury may be at the root cause of catastrophic myocardial damage.

Beyond revascularization many regenerative platforms have been used to try to attenuate myocardial injury after acute myocardial infarction (AMI). Initial interventions targeting cardioprotection focused on activation of the potassium ATP channel in an effort to augment native cardioprotective mechanisms. Beyond cardioprotection, cell-therapeutic efforts to improve outcomes at the time of acute myocardial infarction have been pursued delivering bone marrow mononuclear cells, mesenchymal stem cells and lineage specified cells to the myocardium. Beyond cell-based therapies, gene encoded therapies have been increasingly considered in both heart failure and myocardial infarction. Furthermore, RNA and DNA platforms have been used to deliver VEGF into the myocardium via direct epicardial injection. Furthermore, small interfering RNA (siRNA) and non-encoding microRNA (miRNA) have also been increasingly suggested as potential therapeutic platforms to alter the myocardial microenvironment post-AMI. A barrier towards the realization of these gene technologies is a lack of complementarity with current practice routines. Thus, although pre-clinical efforts have successfully demonstrated biopotency, the translatability of such platforms has remained quite poor.

The M³RNA platform described herein presents a novel approach that is complementary with the current interventional practice, introducing modified mRNA for increased stability, expression, and reduced immunogenicity in vivo. M³RNA complexes were created by microencapsulating modified mRNA in metallic nanoparticles.

Given the complex nature of the post-injury myocardial microenvironment, single gene expression within the heart is likely insufficient achieve any mitigating impact on cardiovascular morbidity. The M³RNA platform is compatible with simultaneous gene delivery of multiple heterologous genes. Furthermore, in certain embodiments, M³RNA biopotentiation of alginate to target the infarcted bed provides a unique opportunity to achieve rapid gene expression in the setting of acute myocardial infarction. To this end, one can envisage delivery of complementary genes serving the angiogenic, cytoprotective, and immunomodulatory needs of the myocardium post-infarction.

The M³RNA platform can target cell survival, impede inflammatory pathways, and act rapidly after restoration of blood flow. However, given that these pathways change within a 48-72-hour period, long-term expression may not be of significant benefit and may pose a risk of harm. Thus, it may be beneficial in certain circumstances that expression of the heterologous gene decreases to some degree after, for example, 72 hours (e.g., 144 hours).

The spontaneous crosslinking of alginate in the presence of Ca′ at the infarcted site provides localized in situ alginate matrix for encapsulating therapeutic RNA for treatment of infarction. The M³RNA platform may be combined with in situ alginate gel formation for targeted gene delivery and expression in acutely infarcted heart to achieve targeted and significant protein expression in three days. This approach could be beneficial for patients suffering from heart attack to achieve rapid, transient, and targeted protein expression within the heart.

Thus, the M³RNA platform serves as a novel technique that would allow interventional delivery of genes immediately after percutaneous coronary intervention with a time horizon tailored to acute events. Beyond the heart, as this technology can induce gene expression in any cell phenotype, the M³RNA platform may be used in other acute events such as musculoskeletal injury, stroke, and sepsis.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Cell Lines and Primary Cell Cultures

Human dermal fibroblasts (HDF), human cardiac fibroblasts (HCF), and human embryonic kidney cells 293 (HEK 293T cells, ATCC CRL-1573) were maintained and passaged in DMEM (with glucose), 10% FBS, 1% pen/strep and 1% glutamine. Initial plating density of cell lines was 200,000 HEK cells and 350,000 HDF and HCF cells/well in 6-well plates. All cell lines were checked periodically for mycoplasma contamination. Time pregnant rats were purchased from Charles River and rat cardiomyocytes were obtained from 19-day-old embryos and cardiomyocytes were isolated according to manufacturer instructions using a neonatal primary cardiomyocyte isolation kit (ThermoFisher Scientific, Waltham, Mass.).

### Antibodies, Messenger RNAs, and Transfections

Antibodies used are anti-mCherry (Rat IgG2a Monoclonal, 1:1000; ThermoFisher Scientific, Waltham, Mass.), anti-Cardiac Troponin T (Mouse IgG1 Monoclonal, 1:200, ThermoFisher Scientific, Waltham, Mass.), anti-FLuc (Goat Polyclonal; 1:250, Novus Biologicals, Littleton, Colo.). EGFP, mCherry and Firefly Luciferase (FLuc) messenger RNAs (Trilink Biotechnologies; San Diego, Calif.) featured modifications such as an anti-reverse cap analog (ARCA cap), polyadenylated tail, and modified nucleotides 5-methyl cytidine and pseudouridine (FIG. 9). In vitro transfection studies for all the cell lines were carried out at approximately 60-65% confluent cells using LIPOFECTAMINE MessengerMAX transfection reagent (ThermoFisher Scientific, Waltham, Mass.). 2.5 μg of indicated mRNA was used per well in 6-well dishes for single transfection or co-transfections. For mice studies, 12 μg of indicated mRNA was used for intra-cardiac injections in mice; 250 μg mCherry mRNA/pig was used for porcine studies.

### Flow Cytometry

Transfection efficiency was determined using FACS Canto (BD Biosciences, San Jose, Calif.). Briefly, cells were mock-transfected or mCherry-mRNA-transfected, trypsinized, and collected at 4 hours and 24 hours (1×10⁶ cells/ml) in 4% formaldehyde in clear polystyrene tubes fitted with a cell filter. Tubes were then introduced into the FACS CantoX for analysis.

### Calcium Imaging

Calcium transients in cardiomyocytes were visualized using CAL-520 AM (AAT Bioquest, Inc., Sunnyvale, Calif.) as previously described (Singh, et al., 2014, J Physiol (Lond) 592:4051). Briefly, cardiomyocytes were transfected with mCherry mRNA overnight and were assessed if the cardiomyocytes were beating post transfection under the microscope. On the following day, cells were loaded with CAL-520 AM (5 mM) 1:1 with POWELOAD (Invitrogen, Carlsbad, Calif.) at the final concentration of 10 μM in Tyrode buffer (in mM) 1.33 CaCl₂, 1 MgCl₂, 5.4 KCl, 135 NaCl, 0.33 NaH₂PO₄, 5 glucose and 5 HEPES. Cells were incubated for 30 minutes in incubator, washed and further incubated for 15 minutes to allow complete de-esterification of Cal-520 AM. Complete medium was added to cells and imaging was performed on Zeiss upright LSM5 live confocal microscope using 20× objective (NA 0.8) in 37° C. humidified chamber with 5% CO₂. Transfected and non-transfected cells in the same area were identified in the mCherry 543 nm excitation. Ca²⁺ transients in rat primary cardiomyocytes were collected at 488 nm excitation. 250 single image frames were collected at 10 fps and the data was analyzed measuring the emitted fluorescence from regions of interest (ROI) over single cardiomyocytes using Zen software and exported to excel and the graphs were created to show Ca²⁺ transients.

### Patch Clamp Recording in Primary Cardiomyocytes

Patch clamp recording was performed with the modification of the protocol previously described (Alekseev et al., 1997, J Membr Biol 157:203; Pitari et al., 2003, Proc Natl Acad Sci USA 100:2695). Neonatal rat primary cardiomyocytes were transfected with mCherry-modified mRNA using the whole-cell configuration of the patch-clamp technique in the voltage-clamp mode. Patch electrodes, with 5-7 MΩ resistance, were filled with 120 mM KCl, 1 mM MgCl₂, 5 mM EGTA, and 10 mM HEPES with 5 mM of ATP 9 (pH 7.3), and cells were superfused with 136.5 mM NaCl, 5.4 mM KCl, 1 mM MgCl₂, 1.8 mM CaCl₂, and 5.5 mM HEPES plus glucose 1 g/l (pH 7.3). Membrane currents were measured using an Axopatch 200B amplifier (Molecular Devices, LLC, San Jose, Calif.). Cellular membrane resistance and cell capacitance were defined online based on analysis of capacitive transient currents. Series resistance (15-20 MΩ), was compensated by 50-60%, and along with uncompensated cell capacitances were continuously monitored throughout experiments. Current density was obtained by normalizing measured currents to cell capacitance. Protocol of stimulation, determination of cell parameters and data acquisition were performed using BioQuest software (Alekseev et al., 1997, J Membr Biol 157:203; Pitari et al., 2003, Proc Natl Acad Sci USA 100:2695; Nakipova et al., 2017, PLoS ONE 12:e0177469). Experiments were performed at 33° C.±1.8° C.

Image Analysis Imaging of cell lines was performed using either upright Zeiss Axioplan epifluorescence wide field microscope (10× objective, NA 0.3) or LSM780 confocal microscope (40× water objective, NA 1.2). Data for quantitation of fluorescence intensity was then analyzed by importing the figures into Tiff format and analyzed using Image J software. Average fluorescence intensity for the whole image was quantified and plotted. (Burgess et al., 2010, Proc Natl Acad Sci USA 107:12564; Singh et al., 2007, J Cell Biol 176:895)
In Vivo Delivery of FLuc, EGFP and mCherry Modified mRNA

In vivo delivery of was carried out in FVB/NJ mice (18-22 grams, aged 6-8 weeks, The Jackson Laboratory, Bar Harbor, Me.) using modified protocol (Yamada et al., 2015, J Am Heart Assoc 4:e001614). Under anesthesia, the heart was exposed and the indicated M³RNA at 12.5 μg/mRNA/mouse (as indicated) was injected in the myocardium of left ventricle. Animals were then imaged or processed for immunohistochemistry at indicated times. 20 animals received M³RNA injections; 10 animals were used for controls.

### Injectable Alginate M³RNA Preparation

Calcium cross-linked alginate solution was prepared by mixing 1 ml of 2% alginate (FMC Corporation, Philadelphia, Pa.) with 0.5 ml of 0.6% Ca gluconate (Sigma-Aldrich, St. Louis, Mo.) and 0.5 ml of water were mixed to yield 2 ml of alginate solution. 500 μl of encapsulated mCherry mRNA (250 μg/pig) was prepared using Nanoparticle in vivo transfection reagent (Altogen Biosystems, Las Vegas, Nev.) according to manufacturer instructions. Solutions were mixed together and injected intracoronary in porcine heart as described below.

### M³RNA Expression in Porcine Myocardial Infarction

Four Yorkshire pigs underwent myocardial infarction using a 90-minute balloon occlusion of the left anterior descending coronary artery. An intracardiac echocardiography (ICE) probe was placed in the right atrium for real-time LV monitoring. Using an AR-2 style coronary catheter the left main artery of the pig was accessed and visualized via fluoroscopy with instilment of Omnipaque. A 0.014″ balanced middleweight coronary wire was advanced into the distal left anterior descending artery (LAD). Utilizing stored guiding angiographic imaging, a 2.5-3 mm balloon was advanced to be positioned across the second diagonal vessel of the LAD. The balloon was inflated to occlude the LAD for 90 minutes followed by reperfusion. Ischemic damage was monitored by ICE as well as continuous ECG telemetry. Following reperfusion, a perfusion catheter was placed at the location of the balloon. Encapsulated mRNA combined with an alginate solution was introduced into the LAD over a 5-minute period and infarct zone targeted gene delivery was documented at day 3.

### Statistics

Data are expressed as Mean±SEM. Statistical significance was determined by GraphPad Prism 7 using One-way or two-way Anova with multiple comparisons. P values less than 0.05 were taken as a statistically significant difference. The ‘n’ values refer to the number of times experiments repeated or the number of animals.

### Example 2

### Materials

Human dermal fibroblasts (HDF), human cardiac fibroblasts (HCF), and human embryonic kidney cells 293 (HEK 293T cells) were maintained and passaged in DMEM (with glucose), 10% FBS, 1% pen/strep and 1% glutamine. Both cell lines were checked periodically for mycoplasma contamination.

mCherry messenger RNA was obtained from Trilink Biotechnologies (San Diego, Calif.). This mRNA was modified using an ARCA cap, polyadenylated tail, and modified nucleotides 5-methyl cytidine and pseudouridine (FIG. 9B).

Modified mRNA (M²RNA) was microencapsulated using MessengerMAX LIPOFECTAMINE (ThermoFisher Scientific, Waltham, Mass.) as an in vitro transfection reagent. M²RNA microencapsulated using a transfection carrier reagent such as MessengerMAX is referred to as microencapsulated modified mRNA (M³RNA).

### Methods

In vitro transfection studies for all the cell lines were carried out using MessengerMAX (ThermoFisher Scientific, Waltham, Mass.). 2.5 μg of indicated mRNA was used per well in 6-well dishes for single transfection or co-transfections. Light phase and fluorescence image of fibroblast cells following modified mRNA transfection were obtained at four hours, 24 hours, 48 hours, and 144 hours; analyses were performed to quantitate the intensity levels of expression at each of those time points. Cells were imaged live in a 37° C. humidified chamber with 5% CO₂. Imaging was performed using either upright Zeiss Axioplan epifluorescence wide field microscope (10×, NA 0.3) or LSM780 confocal microscope (40×/W, NA 1.2). Data for quantitation of fluorescence intensity was then analyzed by importing the figures into Tiff format and analyzed using Image J. Average fluorescence intensity for the whole image was quantified and plotted. Plots were generated based on mean±SEM of average fluorescence intensity (arbitrary units) at indicated time points (n=3). One-way ANOVA with multiple comparisons was performed for statistical analysis. (**** p<0.0001).

Samples at four hours and 24 hours were sorted by mCherry expression to quantitate expression levels and measure the transfection efficiency in these cells. Transfection efficiency was determined using FACS CantoX. Cells were mock-transfected or mCherry-mRNA-transfected, trypsinized, and collected at four hours and 24 hours (10⁶ cells/ml) in 4% formaldehyde in clear polystyrene tubes fitted with a cell filter. Tubes were then introduced into the FACS CantoX for analysis. The efficiency was compared to mock transfected cells as controls. Results are plotted as mean±SEM of 3 different sets of experiments for percent transfection efficiency (n=3) of 3 different cell lines. One-way ANOVA with multiple comparisons was performed for statistical analysis. (**** p<0.0001; ** p<0.01).

Results are shown in FIG. 1 and show that M³RNA can be sustainably expressed in dermal fibroblasts, cardiac fibroblasts, and epithelial cells.

### Example 3

### Materials

Cardiomyocytes were isolated from 19-day-old embryos obtained from pregnant rats (Charles River International, Inc., Wilmington, Mass.). The cardiomyocytes were isolated using a neonatal primary cardiomyocyte isolation kit (ThermoFisher Scientific, Inc., Waltham, Mass.) according to the manufacturer's instructions.

mCherry M²RNA as described in Example 2 was also used.

### Methods

Cardiomyocyte-enriched cultures were verified by documentation of a synchronous beating pattern. These cells were transfected using LIPOFECTAMINE MessengerMAX transfection reagent (ThermoFisher Scientific, Waltham, Mass.) with 2.5 μg of mRNA/well in 6-well dishes for single transfection or co-transfections. Light phase and fluorescence image of cardiomyocytes following M³RNA transfection were obtained at four hours, 24 hours, 48 hours, and 144 hours; analyses were performed to quantitate the intensity levels of expression at each of those time points. Cells were imaged live in a 37° C. humidified chamber with 5% CO₂. Imaging was performed using either upright Zeiss Axioplan epifluorescence wide field microscope (10×, NA 0.3) or LSM780 confocal microscope (40×/W, NA 1.2). Data for quantitation of fluorescence intensity was then analyzed by importing the figures into Tiff format and analyzed using Image J. Average fluorescence intensity for the whole image was quantified and plotted. Quantitation upon transfection (n=3 with >10 images/time point) was plotted as mean±SEM average fluorescence intensity. One-way ANOVA with multiple comparisons was performed for statistical analysis. (**** p<0.0001; *** p<0.001).

Samples at four hours and 24 hours were sorted by mCherry expression to quantitate expression levels and measure the transfection efficiency in these cells. Transfection efficiency was determined using FACS CantoX. Cells were mock-transfected or mCherry-M³RNA transfected, trypsinized, and collected at four hours and 24 hours (10⁶ cells/ml) in 4% formaldehyde in clear polystyrene tubes fitted with a cell filter. Tubes were then introduced into the FACS CantoX for analysis. The efficiency was compared to mock transfected HEK293 cells as controls. Values for the percent transfection from three different sets of experiments were used.

Results are shown in FIGS. 2A-2C and show that M³RNA can be sustainably expressed in cardiomyocytes.

### Example 4

### Materials

Cardiomyocytes were isolated as described in Example 3. EGFP messenger RNA, mCherry messenger RNA, and firefly luciferase (FLuc) messenger RNA were obtained from Trilink Biotechnologies (San Diego, Calif.) and modified as described in Example 2.

### Methods

Cardiomyocyte-enriched cultures were verified and transfected as described in Example 3.

Cells were imaged live in a 37° C. humidified chamber with 5% CO₂. DAPI was used to show cellular nuclei. Imaging was performed using either upright Zeiss Axioplan epifluorescence wide field microscope (10×, NA 0.3) or LSM780 confocal microscope (40×/W, NA 1.2).

Results are shown in FIGS. 2D and 2E and show that multiple M³RNAs can be simultaneously co-expressed in the same cardiomyocytes.

### Example 5

### Materials

FVB/NJ mice aged 6-8 weeks were obtained from Jackson Laboratory, Bar Harbor, Me.

mCherry

mCherry M²RNA was as described in Example 2. Firefly luciferase-containing mRNA (Trilink Biotechnologies, San Diego, Calif.) was used to prepare FLuc M³RNA. The firefly luciferase-containing RNA contained a clean cap and polyadenylation and is, therefore, considered to be an M²RNA.

FLuc M³RNA and mCherry M³RNA were prepared using a nanoparticle-based in vivo transfection reagent (Altogen Biosciences, Las Vegas, Nev.).

FLuc M²RNA was formulated for tail vein injection by mixing 20 μg FLuc M²RNA with 1800 μl of a hydrodynamic solution (Minis Bio LLC, Madison, Wis.).

FLuc M³RNA and mCherry M³RNA were formulated for subcutaneous injection using 20 μg M³RNA with 1800 μl of polyethyleneimine (Polyplus Transfection SA, Illkrich-Graffenstaden, France)

### Methods

Mice were administered a solution of Fluc M²RNA via hydrodynamic tail vein injection or administered mCherry M³RNA or Fluc M³RNA via subcutaneous injection. The amount of luciferase expressed was evaluated at the beginning of the experiment and at two hours, four hours, six hours, and 24 hours after administration. For mice administered nanoparticles containing luciferase mRNA via subcutaneous injection, the amount of luciferase expressed was evaluated at two hours, four hours, six hours, 24 hours, 48 hours, and 72 hours after administration. Mice were administered mCherry M³RNA via subcutaneous injection. mCherry expression was evaluated using fluorescent microscopy.

Luciferase expression was imaged using a Xenogen (IVIS) imaging system. For mice administered a solution of luciferase mRNA via hydrodynamic tail vein injection.

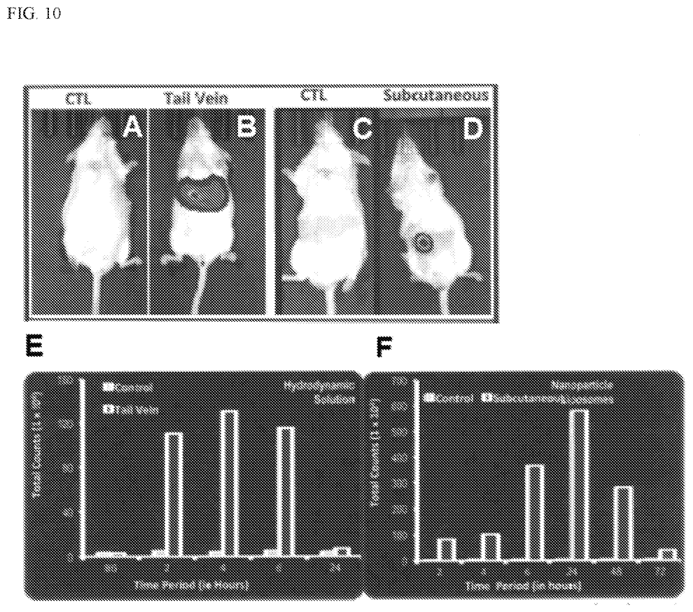

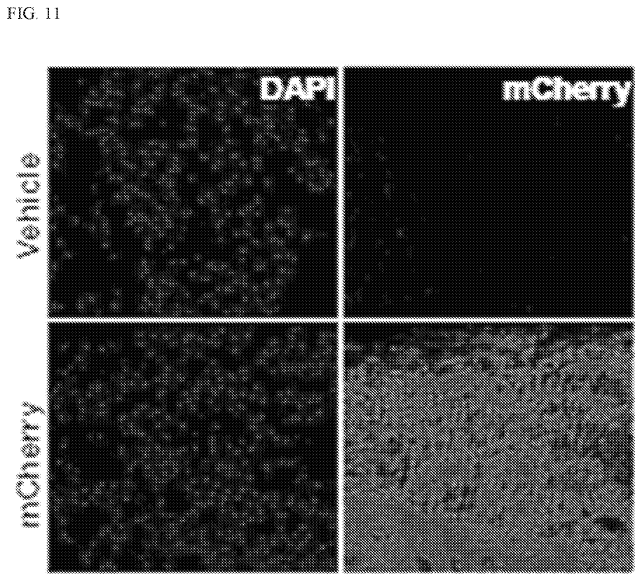

Results are shown in FIG. 10 and FIG. 11 and show that M³RNA can be sustainably expressed in vivo following subcutaneous administration.

### Example 6

### Materials

FVB/NJ mice aged 6-8 weeks were obtained from Jackson Laboratory, Bar Harbor, Me. Firefly luciferase (FLuc) M²RNA was as described in Example 5. M³RNA was prepared with Altogen nanoparticle reagent, as described in Example 5.

### Methods

mCherry and FLuc M³RNA was prepared for administration as described in Example 5. For delivery, either 12 μg mRNA was delivered or a saline volume equivalent by sterile injection. Mice received injections in either the hindlimb, the kidney, or the liver of the mouse. In the case of ocular injection, only 5 μg mRNA was delivered or a saline volume equivalent by sterile injection into the anterior chamber of the eye. All mice were subsequently imaged at multiple times by injecting D-Luciferin intraperitoneally as substrate and evaluating with a Xenogen (IVIS) imaging system.

Results are shown in FIGS. 12A-12D and show that M³RNA can be sustainably expressed in vivo in different organs after direct administration.

### Example 7

### Materials

FVB/NJ mice aged 6-8 weeks were obtained from Jackson Laboratory, Bar Harbor, Me. Luciferase (FLuc) M³RNA was prepared as described in Example 5.

### Methods

12 μg/mRNA/mouse was injected in the myocardium of left ventricle via echo-guided intracardiac injection. Luciferase expression was imaged using a Xenogen (IVIS) imaging system. The amount of luciferase expressed was evaluated at two hours, four hours, six hours, 24 hours, 48 hours, and 72 hours after administration. Data for quantitation of fluorescence intensity was then analyzed by importing the figures into Tiff format and analyzed using Image J. Average fluorescence intensity for the whole image was quantified and plotted.

Results are shown in FIGS. 13A-13B and show that M³RNA can be sustainably expressed in vivo following intracardiac administration.

### Example 8

### Materials

FVB/NJ mice aged 6-8 weeks were obtained from Jackson Laboratory, Bar Harbor, Me. Luciferase (FLuc) M³RNA was prepared as described in Example 5.

### Methods

Under anesthesia, the hearts of the mice were exposed and 12 μg/mRNA/mouse FLuc M³RNA were injected in the myocardium of left ventricle. Animals were then imaged or processed for immunofluorescence at indicated times. Luciferase expression was imaged using a Xenogen (IVIS) imaging system.

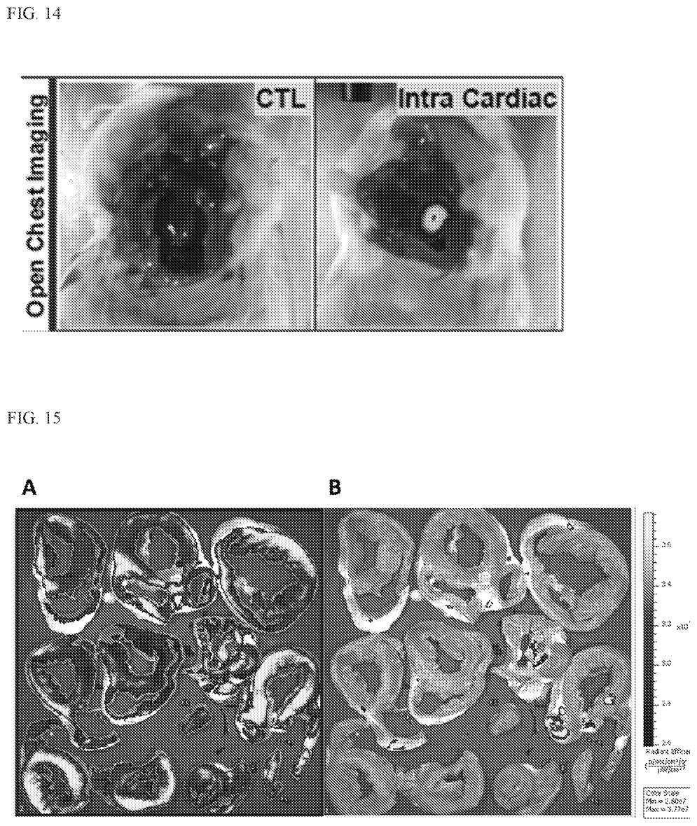

Results are shown in FIGS. 14A-14B and show that M³RNA can be sustainably expressed in vivo following intracardiac administration.

### Example 9

Materials Pharmaceutical grade, high-G alginate (NOVAMATRIX, FMC Biopolymer AS, Sandvika, Norway) and calcium gluconate (0.6% calcium concentration, Sigma-Aldrich, St. Louis, Mo.) were obtained from commercial sources.

mCherry M³RNA was prepared as described in Example 5.

### Methods

The mCherry M³RNA was mixed with alginate for intracoronary delivery. The resulting macroencapsulated alginate solution is referred to as M⁴RNA. For this purpose, a 2% alginate solution (by weight) was first made with RNase-free/DNase-free water. A calcium cross-linked alginate solution (1% alginate) was then prepared by mixing 1 ml of the 2% alginate solution with 0.5 ml of calcium gluconate and 0.5 ml of water to 2 ml of solution. At the time of treatment, 500 μl of mCherry M³RNA (containing 250 μg mRNA) was mixed with 2 ml calcium alginate solution for injection in each pig.

Four adult Yorkshire pigs underwent myocardial infarction using a 90-min balloon occlusion of left anterior descending coronary artery. An intracardiac echocardiography (ICE) probe was placed in the right atrium for real time LV monitoring. Using an AR-2 style coronary catheter, the left main artery of the pig was accessed and visualized via fluoroscopy with instillment of Omnipaque. A 0.014″ balanced middleweight coronary wire was advanced into the distal LAD. Utilizing stored guiding angiographic imaging, a 2.5-3 mm balloon was advanced to be positioned across the second diagonal vessel of the LAD. The balloon was inflated to occlude the LAD for 90 minutes followed by reperfusion. Ischemic damage was monitored by ICE as well as continuous ECG telemetry.

Following reperfusion, a perfusion catheter was placed at the location of the balloon. M4RNA was introduced into the LAD of two of the pigs over a five-minute period and infarct zone targeted gene delivery was documented at day 3 (72 hours). At that point, the heart was harvested, flushed with chilled normal saline and sliced using the ProCUT sampling tool. The amount of mCherry expression was evaluated in the prepared tissues.

Statistical significance was determined by GraphPad Prism 7 using one-way or two-way Anova with multiple comparisons. P values less than 0.05 were taken as a statistically significant difference.

Results are shown in FIGS. 8B and 8C and shows that alginate-based delivery of M⁴RNA with an alginate concentration of 1% results in targeted expression of M³RNA in infarcted cardiac tissue of the pig up to 72 hours after delivery.

### Example 10

### Materials

Pharmaceutical grade, high-G alginate, calcium gluconate, mCherry M³RNA are all as described in Example 9.

### Methods

The M3RNA was mixed with alginate for intracoronary delivery. Two different calcium cross-linked alginate solutions were used: 1.5% alginate concentration and 0.5% alginate concentration. 0.5 ml of mCherry M3RNA (containing 250 μg of mRNA) was mixed with 2 ml of calcium alginate as described in Example 9.

Two adult Yorkshire pigs underwent myocardial infarction using a 90-min balloon occlusion of left anterior descending coronary artery. An intracardiac echocardiography (ICE) probe was placed in the right atrium for real time LV monitoring. Using an AR-2 style coronary catheter, the left main artery of the pig was accessed and visualized via fluoroscopy with instillment of Omnipaque. A 0.014″ balanced middleweight coronary wire was advanced into the distal LAD. Utilizing stored guiding angiographic imaging, a 2.5-3 mm balloon was advanced to be positioned across the second diagonal vessel of the LAD. The balloon was inflated to occlude the LAD for 90 minutes followed by reperfusion. Ischemic damage was monitored by ICE as well as continuous ECG telemetry.

Following reperfusion, a perfusion catheter was placed at the location of the balloon. Each pig received a different alginate concentration with the same dose of M4RNA, each introduced into the LAD of the pigs over a five-minute period. The infarct zone targeted gene delivery was documented at day 3 (72 hours). At that point the heart was harvested, flushed with chilled normal saline and sliced using the ProCUT sampling tool. The amount of mCherry expression was evaluated in the prepared tissues.

Statistical significance was determined by GraphPad Prism 7 using one-way or two-way Anova with multiple comparisons. P values less than 0.05 were taken as a statistically significant difference.

Results are shown in FIG. 15 and show reduced alginate concentration results in diffuse delivery of biologics and loss of signal.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A composition comprising:
an encapsulating agent; and
a polynucleotide encapsulated with the encapsulating agent, the polynucleotide comprising at least one modification that inhibits degradation of the polynucleotide in cytosol of a cell, the polynucleotide encoding at least one therapeutic polypeptide or at least one therapeutic RNA.

2. The composition of claim 1, wherein the polynucleotide modification comprises a pseudoknot, an RNA stability element, or an artificial 3′ stem loop.

3. The composition of claim 1, wherein the encapsulating agent comprises a metallic nanoparticle.

4. The composition of claim 3, wherein the metallic nanoparticle comprises a plurality of metallic subunits that at least partially surround the polynucleotide.

5. The composition of claim 3, wherein the metallic nanoparticle comprises a plurality of metallic subunits forming a core structure.

6. The composition of claim 4, wherein the plurality of nanoparticles comprises:
the metallic nanoparticle; and
at least one nanoparticle comprising a second material.

7. The composition of claim 3, wherein the metallic nanoparticle comprises a surface modification.

8. The composition of claim 7, wherein the surface modification comprises a biocompatible polymer.

9. The composition of claim 8, wherein the biocompatible polymer comprises a net positive charge.

10. The composition of claim 9, wherein the biocompatible polymer comprises chitosan.

11. The composition of claim 1, wherein the polynucleotide comprises mRNA.

12. A method of introducing a heterologous polynucleotide into a cell, the method comprising:
contacting the cell with a pharmaceutical composition that comprises:
an encapsulating agent; and
a heterologous polynucleotide encapsulated with the encapsulating agent, the polynucleotide comprising at least one modification that inhibits degradation of the polynucleotide when the polynucleotide is in cytosol of a cell; and
allowing the cell to take up the composition.

13. The method of claim 12, wherein the cell takes up the composition by endocytosis.

14. The method of claim 12, wherein the heterologous polynucleotide encodes a therapeutic polypeptide or a therapeutic RNA.

15. The method of claim 12, wherein the cell comprises a cardiac cell, a muscle cell, a fibroblast, or a kidney cell.

16. The method of claim 12, wherein the cell is in vivo.

17. The method of claim 12, wherein the heterologous polynucleotide comprises mRNA.

18. The method of claim 12, wherein the encapsulating agent comprises a metallic nanoparticle.

19. The method of claim 18, wherein the metallic nanoparticle comprises a plurality of metallic subunits that at least partially surround the polynucleotide.

20. The method of claim 18, wherein the metallic nanoparticle comprises a plurality of metallic subunits forming a core structure.

21. The method of claim 19, wherein the plurality of nanoparticles comprises:
the metallic nanoparticle; and
at least one nanoparticle comprising a second material.

22. The method of claim 18, wherein the metallic nanoparticle comprises a surface modification.

23. The method of claim 22, wherein the surface modification comprises a biocompatible polymer.

24. The method of claim 23, wherein the biocompatible polymer comprises a net positive charge.

25. The method of claim 24, wherein the biocompatible polymer comprises chitosan.

26. A method of introducing a therapeutic polypeptide or a therapeutic RNA into a cell, the method comprising:
contacting the cell with a pharmaceutical composition that comprises:
an encapsulating agent; and
a heterologous polynucleotide encapsulated with the encapsulating agent, the heterologous polynucleotide comprising at least one modification that inhibits degradation of the heterologous polynucleotide when the heterologous polynucleotide is in cytosol of a cell, the heterologous polynucleotide encoding the therapeutic polypeptide or the therapeutic RNA;
allowing the cell to take up the composition; and
allowing the cell to express the therapeutic polypeptide or the therapeutic RNA encoded by the heterologous polynucleotide.

27. The method of claim 26, wherein the cell takes up the composition by endocytosis.

28. The method of claim 26, wherein the heterologous polynucleotide encodes a therapeutic polypeptide.

29. The method of claim 26, wherein the cell comprises a cardiac cell, a muscle cell, a fibroblast, or a kidney cell.

30. The method of claim 26, wherein the cell is in vivo.

31. The method of claim 26, wherein the heterologous polynucleotide comprises mRNA.

32. The method of claim 26, wherein the encapsulating agent comprises a metallic nanoparticle.

33. The method of claim 32, wherein the metallic nanoparticle comprises a plurality of metallic subunits that at least partially surround the polynucleotide.

34. The method of claim 32, wherein the metallic nanoparticle comprises a plurality of metallic subunits forming a core structure.

35. The method of claim 33, wherein the plurality of nanoparticles comprises:
the metallic nanoparticle; and
at least one nanoparticle comprising a second material.

36. The method of claim 32, wherein the metallic nanoparticle comprises a surface modification.

37. The method of claim 36, wherein the surface modification comprises a biocompatible polymer.

38. The method of claim 37, wherein the biocompatible polymer comprises a net positive charge.

39. The method of claim 38, wherein the biocompatible polymer comprises chitosan.
